(19) United States
(12) Patent Application Publication
(10) Pub. No.: US 2015/0148810 A1
(43) Pub. Date: May 28, 2015

(54) System for Inserting an Intracorneal Lens

(71) Applicant: Neoptics AG, Hünenberg (CH)

(72) Inventors: Holger Broers, Uplengen-Spols (DE); Werner Berner, Erlinsbach (CH)

(21) Appl. No.: 14/405,080

(22) PCT Filed: Jun. 25, 2013
(86) PCT No.: PCT/EP2013/063181

§ 371 Date: Dec. 2, 2014

(30) Foreign Application Priority Data
Jul. 6, 2012 (EP) 12175329.7

(51) Int. Cl. A61F 2/16; A61B 3/00; A61F 9/008; A61B 3/14

(52) U.S. Cl. CPC A61F 2/1662; A61B 3/14; A61B 3/0025; A61F 9/00834; A61F 9/00812; A61F 2009/00872; A61F 2009/00844
USPC 606/107

(57) ABSTRACT

The present invention relates to a system for inserting an intracorneal lens into an eye, comprising an imaging unit (A) for generating an image of the eye, an image processor (4) for generating a virtual image of the eye with the desired position of a pocket to be cut into the cornea, a laser unit (B) for cutting a pocket into the cornea of the eye, with a control device (13) with which, on the basis of the virtual image of the eye generated by the image processor (4), the pocket can be generated in the cornea of the eye by the laser, an insertion unit (C) for inserting an intracorneal lens into the pocket in the cornea with an optical device (11), wherein the insertion of the intracorneal lens with the optical device can be controlled on the basis of the generated virtual image of the eye, by superposing the virtual image of the eye with the real image of the eye visible through the optical device.

The present invention relates to a system for inserting an intracorneal lens into an eye, and a method and a software product for operating such a system.

Intracorneal lenses are used for correcting visual disorders. In contrast to contact lenses, which are placed onto the eye surface, and in contrast to intraocular lenses, which are implanted into an eve chamber, intracorneal lenses are inserted into a pocket created in the cornea. A lens insertion instrument (applicator) is used for inserting the lenses into these pockets.

Intracorneal lenses differ significantly from contact lenses or intraocular lenses, for example in terms of their dimensions, the lack of holding elements (haptic elements) required in the case of intraocular lenses and in terms of the optical properties thereof. Intracorneal lenses are known from the prior art. Reference is made in an exemplary manner to WO 2009/075685, U.S. Pat. No. 5,628,794, U.S. Pat. No. 5,123,921 or EP-1 001 720 B1.

An intracorneal lens is implanted. with the aid of a multistage process. In order to ensure the correct alignment of the intracorneal lens to be inserted along an axis, preferably the visual axis of the eye, defined in advance, the position of the visual axis is initially determined by shining light into the eye and subsequently applying a marking with biocompatible dye directly on the cornea of the eye. Subsequently the pocket for holding the lens is cut into the cornea on the basis of the marking with the aid of a cutting instrument, for example a microkeratome or a laser, preferably a femtosecond laser. The lens is now inserted. into the pocket in the cornea. using an appropriate instrument. The correct positioning of the intracorneal lens in the pocket is checked and readjusted where necessary with the aid of a corresponding instrument.

Performing all the preceding steps with the highest possible precision is a considerable challenge which, to date, has not vet been solved in a satisfactory manner.

WO 2011/047076 A1 describes a device for determining the position of the visual axis. Here, the visual axis of the eye is aligned on the basis of a reference unit (for example a series of concentric rings) and the position thereof is marked on the cornea using e.g. ink. This document therefore merely describes a certain amount of optimization of a partial step.

WO 03/053228 A1 describes a device for ablating the cornea of an eye. The control unit of a laser is controlled with the aid of an image processing unit. Two images of the eye at different wavelengths are recorded, processed and compared to a reference image which, for example, was recorded in the case of a different head position of the patient. Recording two images under different light irradiation serves for better identification of displacements of the eye position in relation to the reference image. However, this device is not suitable for implanting intracorneal lenses in pockets in the cornea.

SensoMotoric Instruments (SMI, www.surgery-guidance.com) has disclosed a system in which the eye is recorded in a manner analogous to WO 03/053228 A1. The image of the eye is subsequently introduced into a surgical microscope so as to assist the surgeon during the intervention. This system still does not offer complete assistance when implanting an intracorneal lens into a corneal pocket either.

It was an object of the present invention to provide a system for improved implantation of an intracorneal lens into a corneal pocket.

According to the invention, this object is achieved by a system and a method for operating the system in accordance with the independent claims.

In detail, the present invention relates to a system for insertion an intracorneal lens into an eye, comprising

a recording unit for producing at least one record of the eye, comprising an image recording device, a device for fixing the eye socket and optionally a reference unit, for example a light source arranged centrally on a lens of the image recording device, which can be fixated on by the eye while the record is produced;

an image processing unit by means of which the at least one image of the eye recorded by the recording unit is processable and a virtual image of the eye with the desired position of a pocket to be cut into the cornea is producible under computer control;

a laser unit for cutting the pocket into the cornea of the eye, comprising a control device by means of which the production of the pocket in the cornea of the eye by the laser is performable under computer control on the basis of the virtual image of the eye produced by the image processing unit;

an insertion unit for inserting an intracorneal lens into the pocket in the cornea, comprising an optical device, for example a microscope, wherein the optical device allows monitoring of the manual insertion of the intracorneal lens on the basis of the virtual image of the eye produced by the image processing unit and, optionally, on the basis of a virtual image of an instrument for inserting the intracorneal lens by superimposing the real image of the eye visible through the optical device on the virtual image of the eye.

According to the invention, an image of an eye recorded with high precision is processed to form a suitable virtual image which comprises all required information for performing the subsequent steps for implanting an intracorneal lens into a corneal pocket. This virtual image is used in all subsequent steps in order to enable a very precise implantation of an intracorneal lens into a corneal pocket.

In accordance with the present invention, a recording unit is initially used to produce at least one record of the eye to be treated. The recording unit comprises an image recording device such as a conventional digital camera, which is preferably fastened to a tripod. Furthermore, in accordance with a preferred embodiment of the present invention, provision is made for a reference unit, which can be fixated on by the eye to be recorded when the record is produced. By way of example, this may be a light source such as an LED, arranged centrally on a lens of the image recording device. The person to be treated is instructed to fixate exactly on the reference unit during the recording. This enables a precise determination of the visual axis of the eye to be treated. However, according to the invention it is also possible to carry out the at least one recording of the eye without such a reference unit. In this case, the at least one record of the-eye is evaluated by a suitable computer program in order to obtain the required information such as the position of the visual axis of the eye.

Since a patient can generally not independently hold his head sufficiently still during such a recording procedure, the recording unit according to the invention additionally comprises a device for fixing the eye socket. Preferably, this is a head fixing unit, as is known, for example from WO 2011/047076 A1 or the above-described system by SMT. By way of example, the head fixing unit comprises a fixed frame which can be fixed to a stationary point such as a tabletop with the aid of attachment means such as clamps. The frame comprises a chin resting unit and a forehead support unit, which are arranged in such a way that the person to be treated can simultaneously place their chin on the chin resting unit and securely press the forehead against the forehead support unit. Preferably, the two units are adjustable in order to be able to adapt the head fixing unit to different facial shapes. Preferably, the head fixing unit can comprise means for fastening a head, for example leather straps which can be placed around the head and affixed using closures. According to the invention, it is not required, but by all means possible, to record records under light conditions that differ from daylight.

According to the invention, it is particularly preferred for the at least one record of the image to be treated to be produced by means of an Ulbricht sphere. An Ulbricht sphere comprises a hollow sphere, wherein the surface condition brings about diffuse reflection of light irradiated into the sphere by a light source. The light source is preferably situated at an angle of 10-40° in relation to a detector. The indirect illumination provided thereby is very comfortable for the person. to be treated and eases the keeping still of the eye to be recorded. Moreover eye geometries and, in particular, eye axes can be determined efficiently using this preferred. embodiment.

According to the invention, the at least one record of the eye to be treated is particularly preferably made in such a way that the visual axis lies centrally in relation to the entry pupil of the eye. This ensures that the far portion and the near portion of the lens receive the same amount of light energy after the lens has been inserted into the corneal pocket and a contraction of the pupil in the case of incidence of light does not lead to the outer zone of the lens being cutoff.

According to the present invention, a plurality of records, for example 3 to 5 records, of the eye are preferably made, said records being evaluated together by the image processing unit and processed to form a virtual image of the eye.

The at least one produced record of the eye to be treated is transmitted to an image processing unit and processed there. The transmission of the image can be performed with the aid of known data transmission methods, for example by means of a direct connection between. recording unit and image processing unit, such as e.g. a data cable, a wireless connection (e.g. WLAN, Bluetooth) between recording unit and image processing unit, or with the aid of a data storage medium such as a USB stick. To this end, the recording unit and image processing unit need to comprise means for data transmission, such as e.g. USB interfaces, to which a data storage medium such as a USB stick can be connected.

The image processing unit according to the invention comprises appropriate known hardware components which are required for image processing, for example a conventional computer with processor and storage modules. The image processing unit according to the invention produces a virtual image of the eye to be treated. Here, the limbus of the recorded eye is used as reference system. An essential aspect consists of the image processing unit according to the invention incorporating calculation of the position of the corneal pocket to be cut in the virtual image of the eve to be treated and, for example, depicting this in the form of a circle. In the process, the image processing unit should preferably comprise means for compensating a parallax error. When the pocket is cut into the cornea, the eye to be treated is, to certain extent, pressed flat with the aid of an applanator or a laser head. What this can lead to in this state is that the position of the pocket to be cut is no longer arranged centrally in relation to the pupil. The image processing unit of the present invention should then be able to compensate for this so-called parallax error and the deformation of the cornea caused by the applanation, and to take these into account when projecting the pocket position in the virtual image of the eye.

Means, that is to say mathematical methods or corresponding computer programs, for compensating a parallax error are known per se. According to the invention, the image processing unit is operated using an image processing program which is additionally equipped with corresponding program sections for compensating a parallax error.

According to the invention, the system comprises a laser unit for cutting the pocket into the cornea of the eye. Preferably, this is a femtosecond laser, as is conventionally used in ophthalmology. Suitable lasers are described in e.g. US 2003/0014042 A1 or WO2008/072092 A1. The laser unit comprises a control device, by means of which the computer controlled production of the pocket in the cornea of the eye is performable by means of the laser on the basis of the virtual image of the eye produced by the image processing unit.

To this end, the data of the virtual image of the eve must be transmitted from the image processing unit to the control device of the laser. The transmission of the data can be performed with the aid of known data transmission methods, for example by means of a direct connection between image processing unit and laser control device, such as e.g. a data cable, a wireless connection (e.g. WLAN, Bluetooth) between image processing unit and laser control device, or with the aid of a data storage medium such as a USB stick. To this end, the image processing unit and laser control device need to comprise means for data transmission, such as e.g. USB interfaces, to which a data storage medium such as a USB stick can be connected.

The control program usable according to the invention is able to perform the operation of the laser on the basis of the data from the image processing unit according to the invention.

In particular, the data relating to the diameter of the limbus are used according to the invention for exactly aligning the laser head. In addition to the above-described virtual projection of the position of the corneal pocket into the virtual image of the eye, it is possible to take into account further data of the virtual image of the eye, for example features of the iris, for positioning the corneal pocket.

With the aid of the laser unit according to the invention, a very precise production of a pocket in the corner of an eye is possible because the laser, in contrast to conventional lasers, can be electronically controlled very precisely on the basis of the above-described virtual image of the eye to be treated.

After producing the pocket in the cornea, the intracorneal lens is manually inserted into the pocket with the aid of the system according to the invention. Great precision is also required here in order to position the lens exactly centrally in relation to the entry pupil, as mentioned above.

The correct insertion of the lens according to the invention into a pocket in the cornea created therefor can particularly preferably be performed according to the invention using an applicator as described in WO 2011/069907 A1. Explicit, reference is hereby made to the relevant content in WO 2011/069907 A1.

WO 2011/069907 A1 describes an applicator comprising a grip piece and a pre-load unit. The grip piece and pre-load unit can be connected to one another, preferably with the aid of a bayonet closure secured against rotation. The pre-load unit can be equipped with a lens in advance and can be stored in a sterile manner in a storage container. In order to insert the lens, the medical practitioner removes the pre-load unit from the storage unit and connects the pre-load unit to the grip piece. Subsequently, the lens can be inserted into the corneal pocket in the manner described in WO 2011/069907 A1.

Use is preferably made of a kit which comprises a storage unit and a pre-load unit in the interior of the storage unit, wherein the storage unit consists of a waterproof material and can be sealed in a waterproof manner using a plug, and the pre-load unit is equipped. with an intracorneal lens. In order to be able to store the pre-load unit in a sterile manner over a relatively long period of time, it is packaged in a storage unit which protects the pre-load unit from surrounding influences. To this end, the interior of the storage unit is filled with a storage liquid which always covers at least the lens situated in the chamber of the pre-load unit. This may be water; however, a physiological saline solution (NaCl) is preferably used as storage liquid.

The pre-load unit is described in detail in WO 2011/069907 A1. Explicit reference is hereby made to the relevant content in WO 2011/069907 A1. The pre-load unit for inserting lenses into the eye of a human or animal comprises:

a housing with means for fastening the unit to a grip piece, preferably in a manner secure against rotation,

a lens-receiving part arranged on or in the housing and comprising a section that protrudes from the housing and that has preferably exactly two separate leaf-like units which, at least at their ends directed away from the housing, are in releasable contact with each other and there form a chamber for storing an optical lens,

a slide, which is arranged movably in the interior of the housing and can be moved between the leaf-like units of the lens-receiving unit.

After removal from the storage unit, the pre-load unit is placed onto a grip piece which is likewise described in detail, in WO 2011/069907 A1. Explicit reference is hereby made to the relevant content in WO 2011/069907 A1. Typically, this is an elongate tube with a form which ensures a simple hold of the grip piece in the hand. At one end, the grip piece is embodied in such a way that the pre-load unit in the grip piece can be connected to one another in such a way that the pre-load unit can be moved within the grip piece. To this end, the grip piece has a diameter which exceeds the diameter of the part of the pre-load unit to be inserted into the grip piece.

With the aid of the applicator from WO 2011/069907 A1, described above, an intracorneal lens can be inserted into a pocket in the cornea in a simple manner. The insertion method comprises the following steps:

positioning the applicator described above at the desired location of the eye in the correct orientation, preferably in a pocket in the human cornea, such that the center of the optical lens contained in the applicator lies on the visual axis of the eye;

pushing the slide forward by means of a first control element on the grip piece, until the slide comes into contact with the lens without moving the latter, wherein at the same time the leaf-like units of the lens-receiving part are spread apart from each other;

withdrawing the rest of the pre-load unit by a defined distance by means of a second control element on the grip piece, with simultaneous fixing of the slide, as a result of which the lens is released from the applicator.

According to the invention, the insertion of an intracorneal lens into the corneal pocket is performed with the aid of the above-described applicator using an insertion unit. This insertion unit for inserting an intracorneal lens into the pocket in the cornea comprises an optical device, for example a microscope. According to the invention, use can be made of surgical microscopes conventionally used in ophthalmology.

According to the invention, the optical device is used to control and monitor the insertion of the intracorneal lens on the basis of the virtual image of the eye produced by the image processing unit and, optionally, on the basis of a virtual image of the instrument for inserting the intracorneal lens. By way of example, this can be realized with the aid of a surgical microscope which comprises a unit by means of which the virtual image of the eye produced by the image processing unit can be introduced into the beam path of the microscope. Such microscopes are known. Surgical microscopes by Leica are referred to in an exemplary manner.

The unit insertable into the beam path of the microscope needs to be supplied with data of the virtual image which was produced by the above-described image processing unit. As described above in the case of the other units, the data can be supplied with the aid of known data transmission methods, for example by means of a direct connection between image processing unit and the unit for the microscope, such as e.g. a data cable, or a wireless connection (e.g. WLAN, Bluetooth) between image processing unit and the unit for the microscope.

On the basis of the data of the virtual image of the eye to be treated, the virtual image of the eye with the position of the corneal pocket can be depicted in the beam path of the microscope with the aid of the above-described unit. In accordance with a preferred embodiment of the present invention, the unit can additionally also produce a virtual representation of the instrument for inserting the intracorneal lens, for example the above-described applicator, in the beam path of the microscope. To this end, a constant data transmission link between the unit in the microscope and the above-described image processing device is advantageous so that required computing capacity can be provided by the image processing device. However, it is also possible to provide the unit or the microscope with appropriate dedicated hardware components which are able to perform computational operations.

In accordance with the present invention, the eye position is “tracked” while the lens is inserted. There is movement of the eve during the insertion process. According to the invention, this eye movement is registered by the system and the virtual image of the eye projected in the beam path of the optical device is modified in such a way that the image of the eye actually observed by the optical device is always superimposed thereon. This ensures precise insertion of the lens into the corneal pocket.

By way of example, relevant eye features such as the structure of the iris are established when the eye is immobilized. These relevant features of the eye are taken into account during the above-described production of the virtual image of the eye. If there is eye movement during the insertion of the lens, the new position of the eye is determined taking into account these relevant features of the eye. The virtual image of the eye and, optionally, the projection of an applicator can be updated accordingly (for example by means of a Fourier transform) and aligned with the actual observed image of the eye.

According to the invention, the actually observed image of the eye is preferably superimposed on the virtual image of the eve in the optical device, as described above. However, according to the invention it is also possible to realize the superimposition of the actually observed image of the eye on the virtual image of the eve on the monitor of an appropriate electronic instrument (e.g. desktop computer, tablet PC). The person performing the insertion of the lens then monitors their actions on the basis of the monitor display.

The surgeon is assisted significantly during the insertion of the intracorneal lens into the corneal pocket by the virtual representations in the beam path of the microscope.

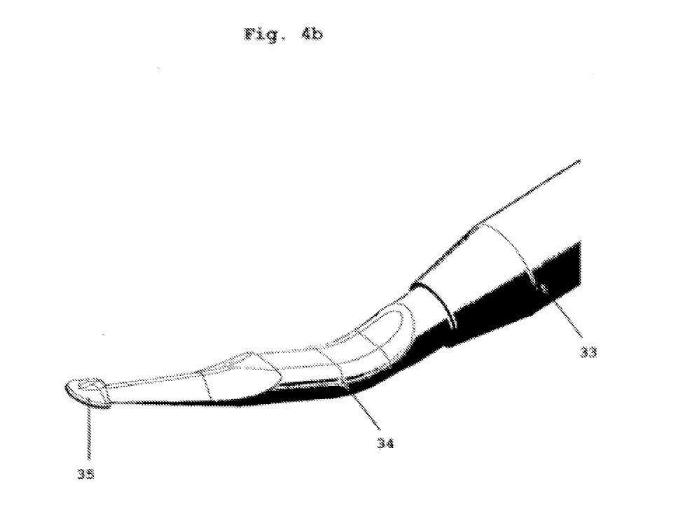

In the case where an intracorneal lens was not inserted precisely into the corneal pocket, a subsequent positioning of the lens can be performed with the aid of a positioning instrument. A positioning instrument as is usable according to the invention is described below in FIGS. 5a and 5b.

Readjusting the position of the intracorneal lens is preferably also performed very precisely using the above-described optical device, for example the above-described microscope, when a virtual image of the eye and, optionally, of the positioning instrument is produced in the beam path of the optical device.

The invention for the first time teaches a system in which all the steps of the implantation of an intracorneal lens into the pocket in the cornea are performed with the aid of a produced virtual image of the eve to tie treated and, optionally, of the positioning instrument (for example the above-described applicator). In this manner, it is possible to achieve a significant increase in the precision of the whole process.

The system according to the invention comprises separate components which, in principle, need not be arranged in the spatial vicinity of one another. Here, “spatial vicinity” is understood to mean a distance between the various system components of at most 10 m. However, according to the invention, it is preferable for at least components b)-d) (i.e. the image processing device, the laser unit and the insertion unit) to be arranged in the spatial vicinity of one another, for example in an operating theater. Furthermore, according to the invention it is preferable for the recording unit not to be arranged in the operating theater if components b)-d) are arranged in an operating theater.

The present invention furthermore relates to a method for operating an above-described system, comprising the following steps:

controlling an image recording device in order to produce at least one record of an eye, preferably when the visual axis of the eye is aligned with a reference unit, for example a light source arranged centrally on a lens of the image recording device;

processing the at least one produced record of the eye in an image processing unit into a virtual image of the eye, wherein the virtual image of the eye comprises the desired position of a pocket to be cut into the cornea;

controlling a control device of a laser unit on the basis of the virtual image of the eye produced by the image processing unit;

providing the virtual image of the eye produced by the image processing unit and, optionally, a virtual image of the instrument for inserting the intracorneal lens in the optical device of the insertion unit for monitoring the insertion of an intracorneal lens in a pocket of a cornea by superimposing the real image of the eye visible through the optical device on the virtual image of the eye.

The present invention furthermore relates to a software product for operating a system as described above, wherein the software product executes the following steps:

controlling an image recording device in order to produce at least one record of an eye, preferably when the visual axis of the eye is aligned with a reference unit, for example a light source arranged. centrally on a lens of the image recording device;

processing the at least one produced record of the eye in an image processing unit into a virtual image of the eye, wherein the virtual image of the eve comprises the desired position of a pocket to be cut into the cornea;

controlling a control device of a laser unit on the basis of the virtual image of the eye produced by the image processing unit;

providing the virtual image of the eye produced by the image processing unit and, optionally, a virtual image of the instrument for inserting the intracorneal lens in the optical device of the insertion unit for monitoring the insertion of an intracorneal lens in a pocket of a cornea by superimposing the real image of the eye visible through the optical device on the virtual image of the eye.

The present invention is explained in more detail below on the basis of non-restrictive drawings and examples. In detail:

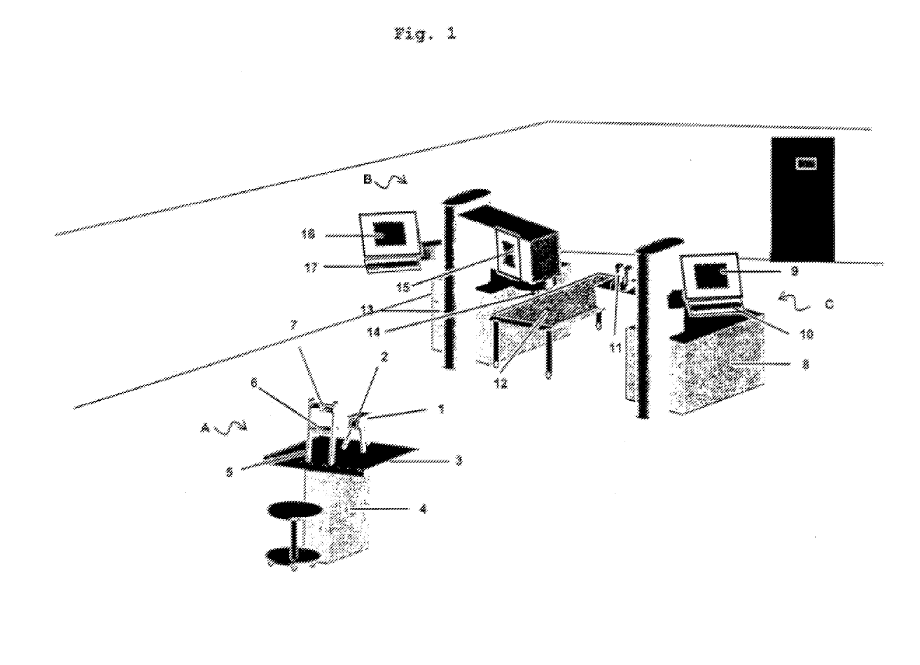

FIG. 1. shows a schematic illustration of the system according to the invention,

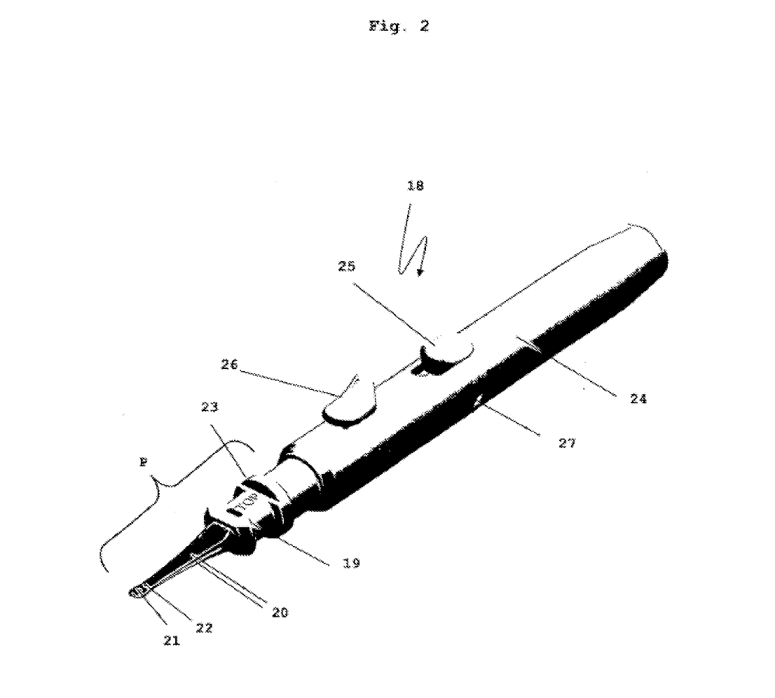

FIG. 2 shows an embodiment. of the applicator in accordance with WO 2011/069907 A1 for inserting the lens according to the invention into a corneal pocket,

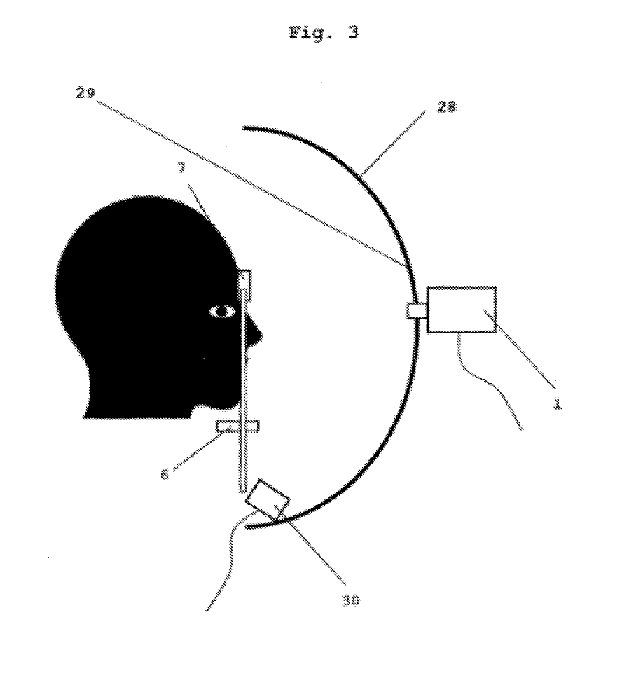

FIG. 3 shows an embodiment of a recording unit according to the invention, based on an Ulbricht sphere,

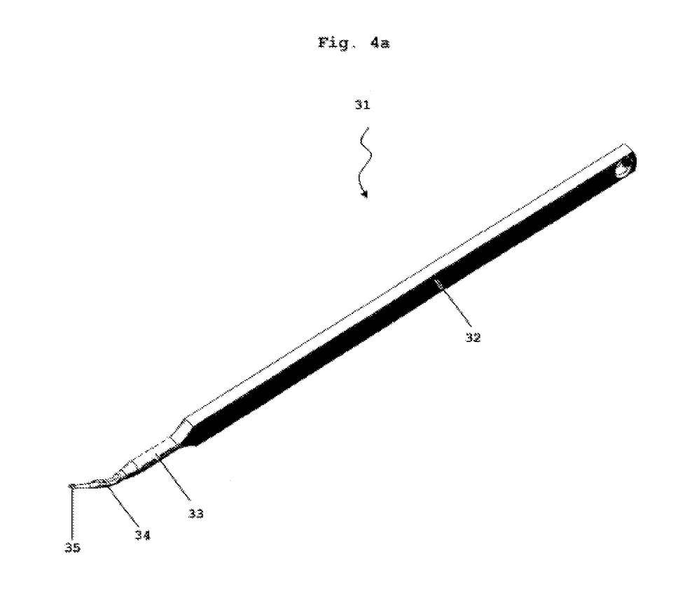

FIG. 4a shows an instrument, usable according to the invention, for positioning the intracorneal lens according to the invention in a corneal pocket, and

FIG. 4b shows a magnified illustration of the positioning head of the instrument in accordance with FIG. 4a.

FIG. 1 shows a schematic illustration of the system according to the invention. The individual components A, B and C are arranged in a room, for example an operating theater. Reference is made to the fact that, according to the invention, this spatial vicinity of the components is very preferred, but not mandatory. As explained above, there are options for transmitting data between the individual components over relatively large spatial distances. However, it is naturally expedient to perform the steps of cutting a pocket into the cornea and inserting an intracorneal lens into this pocket in one room. However, as already explained above, according to the invention it is not preferred for the recording unit to be arranged in the operating theater in this case.

FIG. 1 shows the recording unit A for producing a record of the eye. An image recording device 1, in this case a digital camera with a tripod, is assembled on a tabletop 3. The camera 1 has a lens, on which a reference unit 2 is arranged centrally. in this example, this is a light-emitting diode (LED). A person to be treated can sit down on the chair shown in front of the recording unit A and place their chin on a chin resting unit 6, while simultaneously pressing their forehead against a forehead support unit 7, as a result of which the person fixes their head position for the recording. Together with vertical attachment rods, the chin resting unit 6 and forehead support unit 7 form a device for fixing the eye socket 5, in this case a head fixing unit.

The camera 1 is now used to make at least one record of the eye to be treated, wherein the head of the person to be treated is fixed as described above and the person with the eye to be treated focuses as exactly as possible on the reference unit 2 in the center of the lens of the camera 1. The image data is subsequently supplied to an image processing unit 4 which in the example as per FIG. 1 is arranged in the frame of the tabletop 3. As described above, the data can he transmitted between the recording unit 1 and image processing unit 4 by means of e.g. a direct connection, such as e.g. a data cable, a wireless connection (e.g. WLAN, Bluetooth) or with the aid of a data storage medium such as a USB stick. To this end, the recording unit 1 and image processing unit 4 need to comprise means for data transmission, such as USB interfaces, to which a data storage medium such as a USB stick can be connected.

The image processing unit 4 in accordance with FIG. 1 is a computer with a sufficient computational capacity, on which the image processing program is executed, preferably with modules for compensating a parallax error. With the aid of the image processing unit 4, a virtual image of the eye with the desired position of a pocket to be cut into the cornea is created under computer control. Interface components for operating the image processing unit 4 are generally provided, but not shown in FIG. 1. By way of example, the interface components can be a monitor for displaying data, images etc., and a keyboard or mouse for inputting data and commands.

After at least one record of the eye to be treated has been made, the person to be treated moves to a couch 12. The data relating to the virtual image of the eye to be treated produced by the image processing unit 4 and the calculated position of the lens are transmitted to the control device 13 of the laser unit B, said control device being situated in a housing of the laser unit B. As described above, the data can be transmitted between the image processing unit 4 and laser unit B by means of e.g. a direct connection, such as e.g. a data cable, a wireless connection (e.g. WLAN, Bluetooth) or with the aid of a data storage medium such as a USB stick. To this end, the image processing unit 4 and laser unit B need to comprise means for data transmission, such as USB interfaces, to which a data storage medium such as a USB stick can be connected.

The control device 13 of the laser unit B as per FIG. 1 is a computer with a sufficient computational capacity, on which the control program of the laser is executed. The control device can be operated using conventional interface components. By way of example, FIG. 1 shows a monitor 16 for displaying data, images etc. and a keyboard 17 for entering data and commands. A further monitor 15 may be arranged in the vicinity of the couch 12. The actual laser head 14 is arranged above the couch 12. The component of the laser unit B comprising the laser head 14 is preferably swivelable in order to make space above the couch 12 where necessary. The laser unit B in accordance with FIG. 1 is a femtosecond laser, as is conventionally used in ophthalmology.

As described above, the control program of the laser unit B is able with the aid of the laser to perform the process of cutting a pocket into the cornea under computer control on the basis of the virtual image of the eye to be treated, which image was produced by the image processing unit 4.

Subsequently, an intracorneal lens is manually inserted into the produced pocket in the cornea with the aid of the insertion unit C. The insertion unit C comprises an optical device 11. In accordance with FIG. 1, this is an optical microscope, as is conventionally used in ophthalmology. For the purposes of inserting the lens, the couch 12 with the person to be treated is placed under the optical device 11. This can be brought about either by moving the couch 12 or by pivoting or pulling out the component of the insertion unit C, on which the optical device 11 is situated. The person carrying out the insertion of the lens monitors the insertion of the lens on the basis of the optical device 11 while they perform the actual insertion of the lens with the aid of the above-described applicator, which is still explained in more detail below in FIGS. 2 and 4.

According to the invention, the insertion of the lens is likewise monitored on the basis of the virtual image of the eye to be treated produced by the image processing unit 4. The corresponding data are transmitted to a unit (not shown in FIG. 1) in the beam path of the optical device 11 of the insertion unit C. As described above, the data can be transmitted between the image processing unit 4 and the unit in the beam path of the optical device by means of e.g. a direct connection, such as e.g. a data cable, or a wireless connection (e.g. WLAN, Bluetooth).

In the embodiment in accordance with FIG. 1, provision is made for interface units for monitoring and modifying the virtual image of the eye to be treated projected in the beam path of the optical device 11. By way of example, FIG. 1 shows a monitor 9 for displaying data images etc. and a keyboard 10 for entering data and commands. If computational operations on the virtual image of the eye to be treated are to be performed in the insertion unit C, this can be brought about either by using the image processing unit 4 and transmitting data to the insertion unit C or with the aid of a computer with a sufficient computational capacity which, for example, can be arranged in the housing 8 of the insertion unit C. In accordance with the present invention, the eye position is “tracked” during the insertion of the lens. An eye movement occurs during the insertion process. According to the invention, this eye movement is registered by the system and the virtual image of the eye projected in the beam path of the optical device is modified in such a way that the image of the eye actually observed by the optical device is continuously superimposed thereon. In this manner, a precise insertion of the lens into the corneal pocket is ensured.

After inserting the lens into the pocket of the cornea, there is monitoring with the aid of the optical device 11 of the insertion unit C as to whether the lens is situated precisely at the desired position. In the case where an intracorneal lens was not inserted exactly into the corneal pocket, subsequent positioning of the lens will be performed with the aid of a positioning instrument. A positioning instrument, usable according to the invention, is described in detail below in FIGS. 5a and 5b.

The subsequent adjustment of the position of the intracorneal lens is also performed very precisely with generation of a virtual image of the eye and, optionally, of the positioning instrument in the beam path of the optical device, as described above for the insertion process.

FIG. 2 shows an embodiment of the applicator 18 from WO 2011/069907 A1. A pre-load unit P is movably attached in the grip piece 24. The pre-load unit P comprises a housing 19, two leaf-like units 20 and a stop 23, which restricts the movement of the pre-load unit P into the grip piece 24. In the leaf-like units 20, there is a continuous opening 21 through the center of the chamber (not identifiable here) for receiving a lens. Moreover, provision is made for a further hole 22 which simplifies equipping the applicator 18 with a lens. Two operating elements 25 and 26, with the aid of which the pre-load unit P and a slider (not visible in FIG. 2) can be moved in the interior of the pre-load unit P and of the grip piece 24, are attached to the grip piece 24. A pin, protruding from the lateral opening 27, is fastened to insert parts present in the grip piece and fixes these parts in the grip piece 24. The top side of the pre-load unit P is clearly labeled by the word “TOP”.

FIG. 3 shows an embodiment of a recording unit A according to the invention, based on an Ulbricht sphere. As explained above in relation to FIG. 1, the head of the person to be treated is fixed with the aid of a chin resting unit 6 and a forehead support unit 7. Here, fixing is brought about in such a way that a camera 1 with lens is situated opposite to the eye to be imaged. An Ulbricht sphere 28 which is coated on its inner side 29 with a layer of diffusely reflecting material (e.g. polytetrafluoroethylene (PTFE, Teflon)) is arranged between the camera 1 and chin resting unit 6 and forehead support unit 7. Light from a light source 30 (e.g. an LED) is radiated onto the inner side 29 of the Ulbricht sphere 23 and diffusely reflected there.

FIGS. 4a and 4b depict an instrument, usable according to the invention, for positioning the intracorneal lens according to the invention in a corneal pocket. This positioning instrument may be used when the intracorneal lens is not yet positioned precisely after insertion into a corneal pocket.

The positioning instrument 31 comprises a handle 32 for operating the instrument. A transition element 33 is connected to the handle 32 in an integral or separable manner. The transition element 33 has a bent section 34 which is preferably bent away by an angle of approximately 30° from an imaginary straight line formed by the handle 32 and transmission element 33. As a result, the positioning instrument 31 can be ideally operated as intended by a person. The section 34 can be connected to the transition element 33 in an integral or separable manner. The section 34 is preferably tapered at the end distant from the transition element 33 until it merges into a positioning head 35. The positioning head 35 preferably has a semicircular-shaped form and is connected to the section 34 in an integral or separable manner.

1-15. (canceled)

16. A system for inserting an intracorneal lens into an eye, comprising
a) a recording unit for producing at least one record of the eye, comprising an image recording device, a device for fixing the eye socket and optionally a reference unit which can be fixated on by the eye while the record is produced;
b) an image processing unit by means of which the at least one record of the eye recorded by the recording unit is processable and a virtual image of the eye with a desired position of a pocket to be cut into a cornea is producible under computer control;
c) a laser unit for cutting the pocket into the cornea of the eye, comprising a control device (by means of which a production of the pocket in the cornea of the eye by the laser is performable under computer control on the basis of the virtual image of the eye produced by the image processing unit;
d) an insertion unit for inserting an intracorneal lens into the pocket in the cornea, comprising an optical device, wherein the optical device allows monitoring of a manual insertion of the intracorneal lens on the basis of the virtual image of the eye produced by the image processing unit and, optionally, on the basis of a virtual image of an instrument for inserting the intracorneal lens by superimposing a real image of the eye visible through the optical device on the virtual image of the eye.

17. The system as claimed in claim 16, wherein the desired position of the pocket to be cut into the cornea is implemented by the image processing unit on the basis of the limbus of the eye as reference system.

18. The system as claimed in claim 16, wherein the device for fixing the eye socket is a head fixation unit.

19. The system as claimed in claim 16, wherein the recording unit comprises an Ulbricht sphere.

20. The system as claimed in claim 16, wherein the image processing unit comprises means for compensation of a parallax error.

21. The system as claimed in claim 16, wherein the recording unit and the image processing unit are connected or connectable to one another by means for data transmission.

22. The system as claimed in claim 16, wherein the image processing unit and the laser unit are connected or connectable to one another by means for data transmission.

23. The system as claimed in claim 16, wherein the optical device is a surgical microscope, which comprises a unit by means of which the virtual image of the eye produced by the image processing unit can be inserted into a beam path of the microscope.

24. The system as claimed in claim 23, wherein the unit is directly connected to the image processing unit.

25. The system as claimed in claim 16, wherein the insertion unit additionally comprises an instrument for inserting the intracorneal lens into the pocket in the cornea.

26. The system as claimed in claim 16, wherein the system additionally comprises a device for readjusting a position of the intracorneal lens inserted into the pocket in the cornea.

27. The system as claimed in claim 16, wherein at least components b)-d) are arranged in the spatial vicinity of one another.

28. A method for operating a system, comprising the following steps:
a) controlling an image recording device in order to produce at least one record of an eye;
b) processing the at least one produced record of the eye in an image processing unit into a virtual image of the eye, wherein the virtual image of the eye comprises a desired position of a pocket to be cut into a cornea;
c) controlling a control device of a laser unit on the basis of the virtual image of the eye produced by the image processing unit;
d) providing the virtual image of the eye produced by the image processing unit and, optionally, a virtual image of an instrument for inserting the intracorneal lens in an optical device of an insertion unit for monitoring the insertion of an intracorneal lens in a pocket of a cornea by superimposing a real image of the eye visible through the optical device on the virtual image of the eye.

29. The method as claimed in claim 13, wherein there is compensation of a parallax error when the produced record of the eye is processed into a virtual image of the eye.

30. A software product for operating a system, wherein the software product executes the following steps:
a) controlling an image recording device in order to produce at least one record of an eye;
b) processing the at least one produced record of the eye in an image processing unit into a virtual image of the eye, wherein the virtual image of the eye comprises a desired position of a pocket to be cut into a cornea;
c) controlling a control device of a laser unit on the basis of the virtual image of the eye produced by the image processing unit;
d) providing the virtual image of the eye produced by the image processing unit and, optionally, a virtual image of an instrument for inserting an intracorneal lens in an optical device of an insertion unit for monitoring the insertion of an intracorneal lens in a pocket of a cornea by superimposing a real image of the eye visible through the optical device on the virtual image of the eye.
